(19) United States
(12) Patent Application Publication
(10) Pub. No.: US 2025/0327788 A1
(43) Pub. Date: Oct. 23, 2025

(54) METHOD AND SYSTEM FOR DETERMINING IN-SITU STRESSES IN ANISOTROPIC ROCKS

(71) Applicant: CHINA PETROLEUM & CHEMICAL CORPORATION, Beijing (CN)

(72) Inventors: Jon Jincai ZHANG, Houston, TX (US); Zhihui FAN, Houston, TX (US); Peiqing LIAN, Houston, TX (US); Xueyou HU, Houston, TX (US)

(21) Appl. No.: 19/184,747

(22) Filed: Apr. 21, 2025

Related U.S. Application Data
(60) Provisional application No. 63/636,694, filed on Apr. 19, 2024.

(51) Int. Cl. G01N 33/24; E21B 49/00; E21B 49/02

(52) U.S. Cl. CPC G01N 33/24; E21B 49/005; E21B 49/02

(57) ABSTRACT

Methods and systems for determining in-situ stresses in anisotropic rocks are presented. The methods consider both anisotropic rock properties, geothermal and tectonic effects. They calculates in-situ stresses for subsurface rocks with anisotropies and non-isothermal effects, so that they can be applied to geothermal energy and geo-energy. Horizontal stresses in the vertical transverse isotropy (VTI) rock and in the horizontal transverse isotropy (HTI) rock are obtained for calculating in-situ stresses in naturally fractured rocks. Compared with the conventional isotropic model, the method applicable to VTI rocks predicts a higher minimum horizontal stress and a higher maximum horizontal stress, which is suitable for shales and other laminated formations. The method applicable to HTI rocks gives a lower minimum horizontal stress than the conventional model. Geothermal temperature effects are also integrated into the methods so that the methods are applied to geothermal energy.

### FIELD OF THE INVENTION

The present disclosure relates to methods and systems for determining the in-situ stresses in subsurface rocks, which is applicable in the fields of geo-energy and geological engineering, e.g., geothermal energy, oil and gas exploration and production.

### BACKGROUND

In-situ stresses are critical parameters for geothermal energy and geological operations. For example, in oil and gas exploration, the stress-strain behavior of reservoir rocks is crucial for understanding and predicting mechanical and hydraulic properties of the rocks, such as porosity, permeability, fracture initiation and propagation, and compaction.

in-situ stresses have three principal stresses—the minimum horizontal stress (σₕ), the maximum horizontal stress (σ_{H}), and the vertical stress (σ_{V}), as shown in FIG. 1. Determinations of the minimum and maximum horizontal stresses are difficult. Field tests can be used to measure horizontal stresses. However, field test results may not be available for the interested depth and in new areas. Conventional methods used to estimate horizontal stresses are mainly for isotropic rocks. However, many geologic formations contain anisotropic rocks, e.g., laminated shales and naturally fractured rocks. For these rocks, using conventional methods to calculate the minimum and maximum horizontal stresses produces erroneous results.

Further, existing studies based on rock anisotropies are deficient because they only estimate stresses based on oversimplified models, and often fails to consider temperature effect. New methods are needed to calculate both minimum and maximum horizontal stresses with consideration of rock temperature effects. This disclosure shows the methods for more accurately calculating in-situ stresses in subsurface rocks taking into consideration rock anisotropies and thermal effects.

SUMMARY The disclosure provides a method for determining horizontal stress in a subsurface anisotropic rock. The method includes the steps of
S0: investigating the anisotropic rock by one or more means selected from well logging, seimic survey, and core sampling of the anisotropic rock;
S1: calculating a vertical stress based on a bulk density of the anisotropic rock;
S2: calculating a pore pressure based on well log data or seismic survey data;
S3: conducting laboratory testing of a core sample of the anisotropic rock to obtain parameters including Young's modulus, Poisson's ratio, and thermal expansion coefficient, and tectonic strain;
S4: determining a dip angle of factures in the anisoptropic rock; and
S5: calculating the minimum horizontal stress and/or the maximum horizontal stress in the anisotropic rock according to the dip angle in the anisotropic rock.

According to one aspect of an embodiment in this disclosure, when the dip angle is 0°, calculating the minimum horizontal stress according to Eq. (1), and calculating the maximum horizontal stress according to Eq. (2):

According to another aspect of an embodiment in this disclosure, when the dip angle is 90°, calculating the minimum horizontal stress according to Eq. (3), and calculating the maximum horizontal stress according to Eq. (4).

According to a further aspect of an embodiment in this disclosure, when the dip angle is between 0° and 90°, calculating the minimum horizontal stress of the anisotropic rock according to Eq. (5).

According to still an aspect of an embodiment in this disclosure, the method further includes steps of:
S6: obtaining a measured maximum horizontal stress and a measured maximum horizontal stress; and comparing the measured minimum horizontal stress with the calculated minimum horizontal stress to obtain a first difference and/or comparing the measured maximum horizontal stress with the calculated maximum horizontal stress to obtain a second difference; and
S7: when the first difference or the second difference exceeds a threshold value, adjusting a value of the tectonic strain; otherwise, outputting the calculated minimum horizontal stress and the calculated maximum horizontal stress as true; and
S8: repeating S5 to S7 until the first difference, or the second difference, or both are at or below the threshold value.

### BRIEF DESCRIPTION OF THE DRAWINGS

The teachings of the present invention can be readily understood by considering the following detailed description in conjunction with the accompanying drawings.

FIG. 1 shows in-situ stresses in the subsurface rocks, including vertical stress (σ_{V}), minimum horizontal stress (σₕ), maximum horizontal stress (σ_{H}). Properties of the rocks further include pore pressure (pp) and temperature (T).

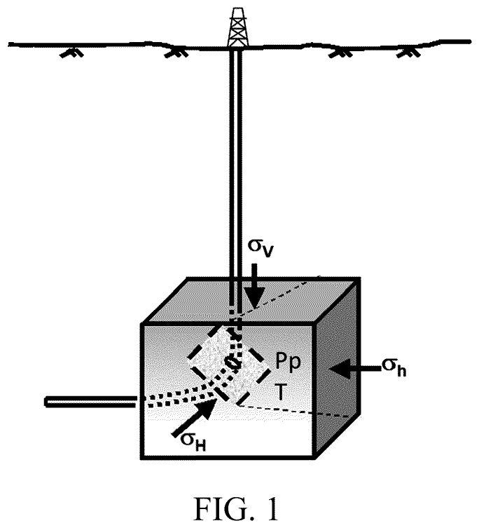

FIG. 2 illustrates a wireline well logging operation in the field.

FIG. 3 is a schematic diagram showing a method of laboratory characterization of a VTI rock specimen having a set of horizontal fractures.

FIG. 4 illustrates a further example of laboratory characterization of a VTI rock specimen.

FIG. 5 is a schematic showing a method of laboratory characterization of a HTI rock specimen having a set of vertical fractures.

FIG. 6 illustrates a further example of laboratory characterization of a HTI rock specimen.

FIG. 7 is the flow chart illustrating methods for determining in-situ stress of anisotropic rocks.

FIG. 8 presents the measured data in 10 wells in the Xu2 formation in the Xinchang gas field. Panel (a) shows fracture density versus the minimum horizontal stress gradient, while panel (b) shows fracture density versus formation breakdown pressure gradient.

FIG. 9 presents the measured data in 13 wells in the Xu2 formation in the Xinchang, Xinsheng and Gaomiao gas fields. Panel (a) shows fracture dip angle versus the minimum horizontal stress gradient, while panel (b) shows fracture dip angle versus formation breakdown pressure gradient.

FIG. 10 compares minimum horizontal stresses calculated using different methods. Panel (a) shows Gamma ray well log data; panel (b) presents fracture dip angles interpreted from well logs; and panel (c) compares the minimum horizontal stresses (Sₕ) estimated from empirical data, a simplified anisotropic model according to Eq. (5), and an isotropic model.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION OF EMBODIMENTS

### 1. Method for Determining Horizontal Stresses Using the Vertical Transverse Isotropy (VTI) Model

According to one aspect of the currently disclosure, many sedimentary rocks, particularly shale oil and gas formations are composed of rocks containing interfaces or bedding planes. This is one of the most common anisotropic effects in the rocks, and it can be modeled using the vertical transverse isotropy (VTI) model. In the VTI model, rock properties (i.e., formation properties) are uniform horizontally within a layer but vary vertically from layer to layer, and the vertical axis is the axis of symmetry, while the horizontal plane is the plane of transverse isotropy. Such a rock is referred to herein as a VTI rock.

For the VTI rocks, Eqs. (1) and (2) can be employed to calculate the minimum and maximum horizontal stresses, which takes into consideration thermal effect (i.e., the effect of temperature in the subsurface rocks):

$${σ}_{h \_ VTI} = \frac{{E}_{h} {v}_{V}}{{E}_{V} ( 1 - {v}_{h} )} ( {σ}_{V} - {α}_{V} {p}_{p} ) + {α}_{h} {p}_{p} + \frac{{E}_{h}}{1 - {v}_{h}^{2}} ( {ε}_{h} + {v}_{h} {ε}_{H} ) + \frac{{E}_{h} {α}_{Th}}{1 - {v}_{h}} Δ T , \qquad ( 1 )$$

$${σ}_{H \_ VTI} = \frac{{E}_{h} {v}_{V}}{{E}_{V} ( 1 - {v}_{h} )} ( {σ}_{V} - {α}_{V} {p}_{p} ) + {α}_{h} {p}_{p} + \frac{{E}_{h}}{1 - {v}_{h}^{2}} ( {ε}_{H} + {v}_{h} {ε}_{h} ) + \frac{{E}_{h} {α}_{Th}}{1 - {v}_{h}} Δ T . \qquad ( 2 )$$

In Eqs. (1) and (2), σ_{h_VTI} and σ_{H_VTI} are the minimum and maximum horizontal stresses in the VTI rocks, respectively; E_{V} and v_{V} are the static Young's modulus and Poisson's ratio in the vertical direction, respectively; Eₕ and vₕ are the static Young's modulus and Poisson's ratio in the horizontal direction, respectively; σ_{V} is the vertical stress; pₚ is the pore pressure; αₕ and σ_{V} are Biot's coefficients in the horizontal and vertical directions, respectively; εₕ and ε_{H} are the tectonic strains in the minimum and maximum horizontal stress directions, respectively; α_{Th} is the thermal expansion coefficient in the horizontal direction; and ΔT is the temperature increase (or decrease) in the burial history of the rocks.

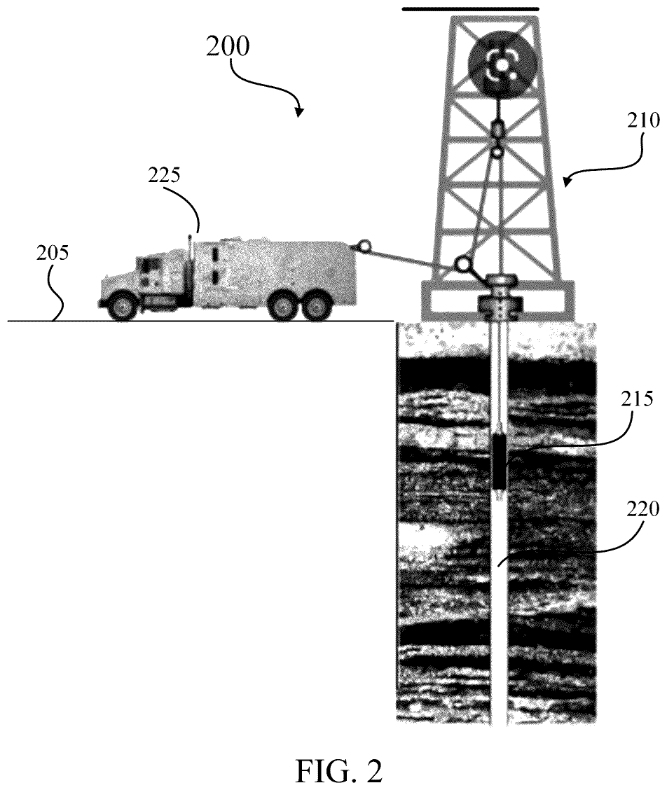

According to Eqs. (1) and (2), when anisotropic rock properties (E_{V}, Eₕ, v_{V}, vₕ, αₕ, α_{V}, and α_{Th}), vertical stress (σᵥ), pore pressure (pₚ), and tectonic strains (εₕ and ε_{H}) are known, the minimum and maximum horizontal stresses in the VTI rocks can be obtained. Dynamic rock properties of the VTI rocks can be calculated from seismic velocities or acoustic velocities from well log measurements through cross dipole sonic method, as shown in FIG. 2. These dynamic properties are converted into static properties before being used in Eqs. (1) and (2).

FIG. 2 is schematic diagram illustrating a cross-sectional view of a seismic survey region with a wellbore and a wireline well logging tool including one or more sonic generator and one or more well log data recording sensors according to an embodiment. A sonic generator is an example of equipment to produce one or more sonic waves (sound waves). A sonic generator may be referred to as a sonic source because the sonic generator produces or generates one or more sonic waves (sound waves) which are also referred to as seismic waves. The one or more well log data recording sensors are examples of one or more seismic data recording sensors (seismic receivers or seismic data recorders) and may be the same seismic data recording sensors as seismic data recording sensors. In embodiments of the present invention, oil and/or gas production is discontinued in order to generate seismic waves and record seismic data including reflections of the seismic waves moving through the subsurface of one or more earth formations in the seismic survey region.

FIG. 2 also shows a drilling system 200 on land 205 including a drilling rig 210. The drilling rig 210 supports the lowering of a wireline well logging tool 215 into a wellbore 220. The well logging tool 215 may include one or more sonic generators (sonic sources) to generate one or more sound waves, which are transmitted into one or more earth formations to generate reflections or reflection waves in the one or more earth formations. Although this example shows one or more earth formations of a land-based survey region, it is understood that this is only an example and that the methods and systems may also be applied to a survey region at the surface or bottom of a body of water such as an ocean.

The well logging tool 215 also includes one or more well log data recording sensors. As discussed above, the one or more well log data recording sensors receive and record well log data, which includes reflection data received by the one or more well log data recording sensors in response to the sound waves transmitted into one or more earth formations by the one or more sonic generators.

The well log data is an example of seismic data. The well log data may include compressional wave velocity or P-wave velocity (Vp), shear wave velocity (Vs), and density, which is an indicator of porosity. This well logging process to record well log data may also be referred to as sonic logging.

Referring again to FIG. 2, a vehicle 225 may be coupled to the well logging tool 215 to assist in the lowering and raising of the well logging tool 215 as well as communicating with the well logging tool 215 to obtain well log data. Alternatively, in methods and systems for a survey region at the surface or bottom of a body of water such as an ocean, another device or system may used to assist in the lowering or raising of the well logging tool 315 as well as communicating with the well logging tool 215 to obtain well log data.

Likewise, well log data can also be obtained through known LWD (logging while drilling) and MWD (measurement while drilling) methods and tools. In this disclosure, MWD generally refers to real-time collection and analysis of data downhole that provides real-time feedback relating to drilling parameters to the operator. LWD generally refers to collecting, storing or transmission of data for analysis. Instead of using the wireline well logging tool 215, LWD and MWD tools are installed on the drilling assembly in the drilling string and advance together with the drilling assembly. At the same time, LWD and MWD tools can measure subsurface formation properties and transmit data to a computer system on the surface for further processing. Using either the wireline logging tool or LWD tools, dynamic rock properties can be obtained, which can be converted into static rock properties for use in Eqs. (1) and (2).

According to another aspect of the current disclosure, static Young's moduli and Poisson's ratios of the VTI rocks (e.g. E_{V}, Eₕ, v_{V}, vₕ) are obtained from laboratory tests by exerting loading stresses in horizontal and vertical directions.

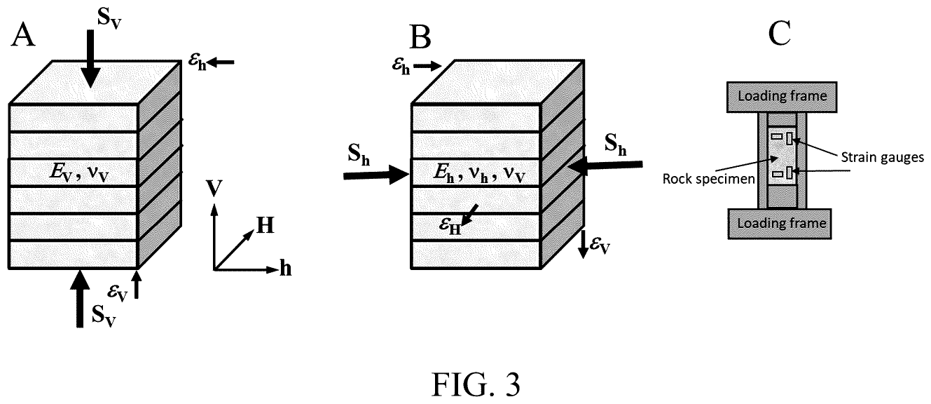

FIG. 3 illustrates the VTI rock having a set of horizontal fractures. Insert A in FIG. 3 illustrates compression tests for obtaining anisotropic Young's moduli and Poisson's ratios in the VTI rock under a loading stress (Sv) in a cubic specimen in vertical direction. In this case, Sv is perpendicular to the planar direction of the horizontal fractures. Insert B in FIG. 3 illustrates the compression tests for obtaining anisotropic Young's moduli and Poisson's ratios in the VTI rock under a loading stress (Sₕ) in a cubic specimen in the minimum horizontal stress direction, i.e., in a direction that parallel to the horizontal factures. Insert C in FIG. 3 is a schematic diagram of the apparatus to conduct compression test, in which the rock specimen is placed between a top loading frame and a bottom loading frame. Tightening the loading frames exerts a load on the specimen and cause strains that are shown in the strain gauges.

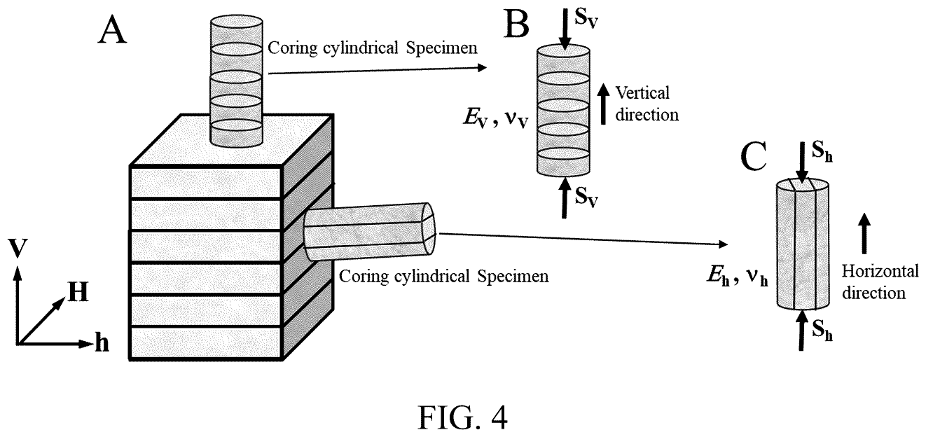

Likewise, FIG. 4 illustrates laboratory characterization of another VTI rock specimen. Insert A shows cylindrical core sample/specimen for laboratory compression tests in the VTI rock with a set of horizontal fractures, aka. bedding planes. Insert B shows the compression tests for obtaining anisotropic Young's modulus and Poisson's ratio (E_{V} and w_{V}) in the vertical cylindrical core sample under a loading stress (S_{V}) in the vertical direction. Insert C illustrates the compression tests for obtaining anisotropic Young's modulus and Poisson's ratio (Eₕ and vₕ) in the horizontal cylindrical core sample under a loading stress (Sₕ) in the horizontal direction.

According to both FIGS. 3 and 4, at least 2 rock specimens and 2 uniaxial compressional loading tests are needed to obtain parameters of E_{V}, Eₕ, v_{V}, vₕ. Based on laboratory test data in more than 10 worldwide shale oil and gas formations, Young's moduli in horizontal direction and in vertical direction have the following empirical relation: Eₕ/E_{V}=1.44.

### 2. Method for Determining Horizontal Stresses Using the Horizontal Transverse Isotropy (HTI) Model

According to a further aspect of the current disclosure, another typical rock formation contains a set of mutually parallel vertical fractures. This type of rock formation is isotropic along the vertical direction but anisotropic in a horizontal direction, which can be described based on horizontal transverse isotropy (HTI) model. This type of rocks are thereby referred to as HTI rocks.

The minimum and maximum horizontal stresses in the HTI rocks with consideration of thermal effect can be expressed in Eqs. (3) and (4):

$${σ}_{h \_ HTI} = \frac{{E}_{h} {v}_{Vh} ( 1 + {v}_{VH} )}{{E}_{V} ( 1 - {v}_{Vh} {v}_{hV} )} ( {σ}_{V} - {α}_{V} {p}_{p} ) + {α}_{h} {p}_{p} + \frac{{E}_{h} ( {ε}_{h} + {v}_{Vh} {ε}_{H} )}{1 - {v}_{Vh} {v}_{hV}} + \frac{{E}_{h} ( {α}_{Th} + {v}_{Vh} {α}_{TH} )}{1 - {v}_{Vh} {v}_{hV}} Δ T , \qquad ( 3 )$$

$${σ}_{H \_ HTI} = \frac{{v}_{VH} + {v}_{Vh} {v}_{hV}}{1 - {v}_{Vh} {v}_{hV}} ( {σ}_{V} - {α}_{V} {p}_{p} ) + {α}_{V} {p}_{p} + \frac{{E}_{V} ( {ε}_{H} + {v}_{hV} {ε}_{h} )}{1 - {v}_{Vh} {v}_{hV}} + \frac{{E}_{V} ( {α}_{TH} + {v}_{hV} {α}_{Th} )}{1 - {v}_{Vh} {v}_{hV}} Δ T . \qquad ( 4 )$$

In Eqs. (3) and (4), σ_{h_HTI} and σ_{H_HTI} are the minimum and maximum horizontal stresses in the HTI rocks, respectively; E_{V} and Eₕ are the static Young's moduli in the vertical and horizontal directions, respectively; v_{Vh}, v_{VH}, and v_{hV} are the static Poisson's ratios in the HTI rocks; v_{hH}=v_{hV}, v_{HV}=v_{VH}, and v_{Vh}=v_{Hh}, and v_{hV}/Eₕ=v_{Vh}/E_{V}; α_{Th} and α_{TH} are the thermal expansion coefficients in the two horizontal directions. Other symbols have the same meanings as those in Eqs. (1) and (2).

According to Eqs. (3) and (4), when the anisotropic rock properties (E_{V}, Eₕ, v_{Vh}, v_{vH}, v_{hV}, αₕ, α_{V}, α_{Th}, α_{TH}), the vertical stress (σ_{V}) and pore pressure (pₚ), tectonic strains (εₕ and ε_{H}) are known, the minimum and maximum horizontal stresses in the HTI rocks can be calculated. The dynamic rock properties of the HTI rocks can be calculated from the seismic velocity or acoustic velocities from well logging measurements through cross dipole sonic method, as shown in FIG. 2.

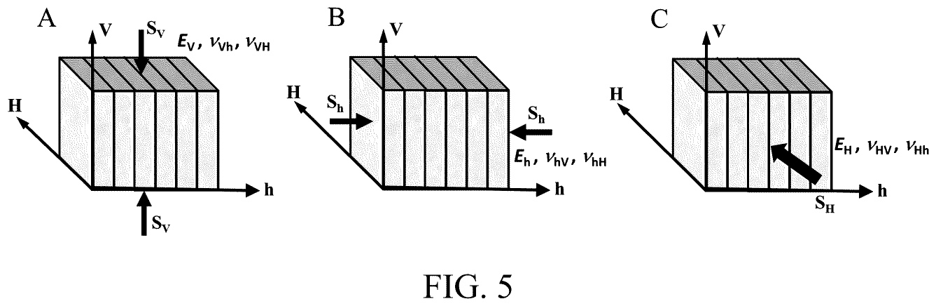

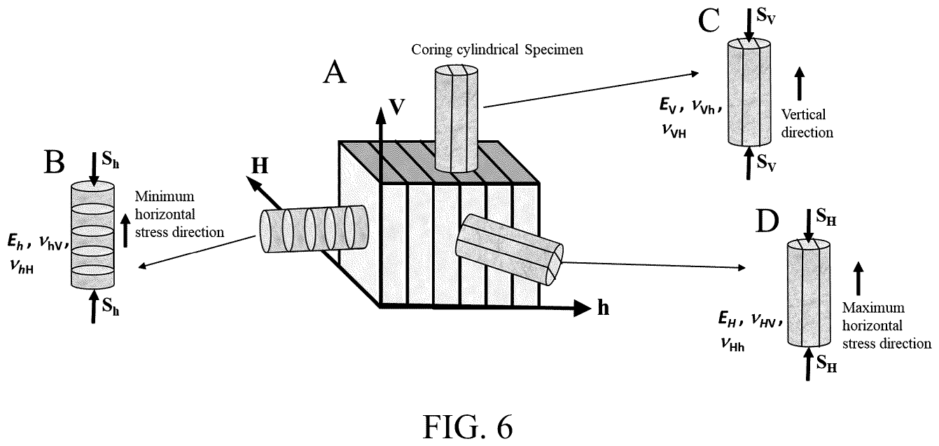

According to a further aspect of the embodiment in this disclosure, static Young's moduli and Poisson's ratios of the HTI rocks (e.g. E_{V}, Eₕ, v_{Vh}, v_{VH}, and v_{hV}) from laboratory tests by exerting loading in vertical and two horizontal directions to rock specimens, as shown in FIG. 5 or 6.

In FIG. 5, insert A illustrates the compression test for obtaining anisotropic Young's moduli and Poisson's ratios in the HTI rock for a loading stress (Sv) along a vertical direction in a cubic specimen. Insert B illustrates the compression tests for obtaining anisotropic Young's moduli and Poisson's ratios in the HTI rock for a loading stress (Sₕ) in the horizontal direction perpendicular to the vertical factures in a cubic specimen, i.e., the minimum horizontal stress direction. Insert C in FIG. 5 illustrates the compression tests for obtaining anisotropic Young's moduli and Poisson's ratios in the HTI rock for a loading stress (S_{H}) in a horizontal direction parallel to the vertical fractures, i.e., the maximum horizontal stress direction.

In FIG. 6, insert A shows a cylindrical core sample for laboratory compression tests in the HTI rock. Insert B illustrates the compression tests for obtaining anisotropic Young's modulus and Poisson's ratios (Eₕ, v_{hV} and v_{hH}) in the cylindrical specimen cored in the minimum horizontal stress direction under a loading stress (Sₕ). Insert C illustrates the compression test for obtaining anisotropic Young's modulus and Poisson's ratios (E_{V}, v_{Vh} and v_{VH}) in the cylindrical specimen cored in vertical direction under a loading stress (Sv). Insert D illustrates the compression tests for obtaining anisotropic Young's modulus and Poisson's ratios (E_{H}, v_{HV} and v_{Hh}) in the cylindrical specimen cored in the maximum horizontal stress direction with a loading stress (S_{H}).

### 3. Method for Determining Horizontal Stresses in the Anisotropic Rocks

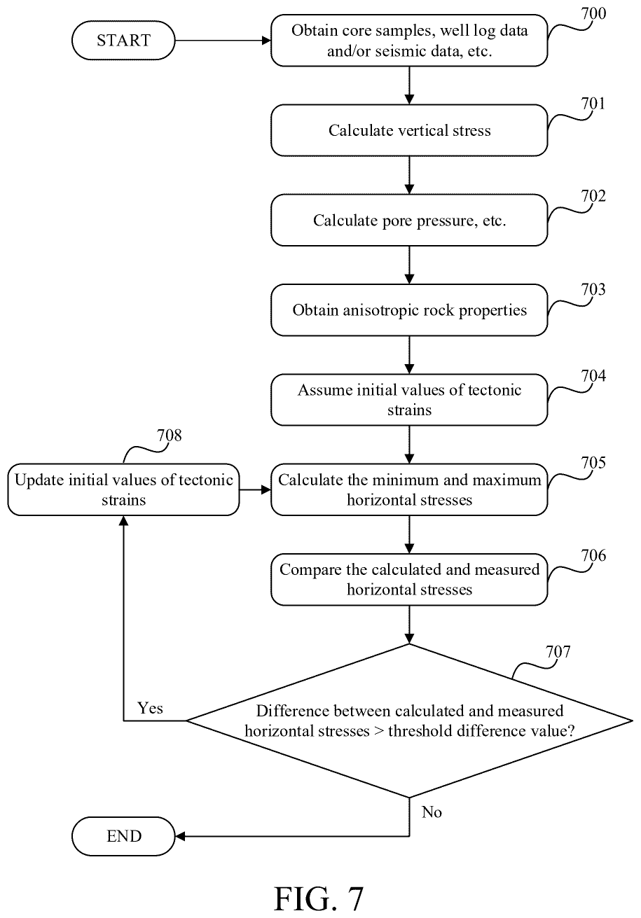

FIG. 7 illustrates the process for obtaining parameters of anisotropic rock properties and calculating horizontal stresses for the VTI or HTI rocks, taking into consideration of the thermal effect.

In Step 700, well log data or seismic data are obtained, e.g., by collecting existing data or by conducting well logging or seismic survey operations. Core samples may also be drilled or otherwise collected.

In Step 701, vertical stress (σ_{V}) is obtained based on bulk density of the anisotropic rock. The bulk density data is obtained based on prior exploration of the area of interest or is obtained by well logging operations (e.g., shown in FIG. 2) when the area of interest has not been investigated previously. Specifically, the snode or the LWD tool may contain a density logging tool. The bulk density can be done by integration of bulk density data measured from bulk density logs in the one or more wells.

In Step 702, pore pressure (pₚ) is obtained using well log data and resistivity data from the nearby wells or using seismic data in the area of interest. Seismic data are obtained from seismic surveys. The well log data, i.e., the seismic interval velocity from a rock formation, in turn is obtaining using a well logging tool installed in the snode or in the LWD tool. The resistivity data is obtained using a resistivity tool in either wireline logging or LWD logging. Pore pressure in the anisotropic rock can be calculated based on the sonic logging data and resistivity data using Eaton's method. The measured pore pressure values in the nearby wells are used to calibrate the pore pressures estimated from the well log data and to improve the method of the pore pressure estimation.

In Step 703, rock properties of the anisotropic rock are obtained by testing rock specimen in the laboratory. The rock properties include Young's moduli, Poisson's ratios, Biot's effective stress coefficients, and thermal expansion coefficients. The Young's modulus and the Poisson's ratio can be measured as shown in FIGS. 3-6. The Biot's effective stress coefficient is also obtained through careful lab testing or is estimated based on seismic data or well log data using known methods. Thermal expansion coefficient is measured in the lab using known methods, e.g., using dilatometry, thermomechanical analysis, and interferometric methods, etc.

In Step 704, initial values of tectonic strains are assigned based on empirical data, e.g., historical data.

In Step 705, the minimum and maximum horizontal stresses are calculated. In this step, the anisotropic rock under investigation is first determined to approximate a VTI rock or a HTI rock. For the VTI rock, the calculation is according to Eqs. (1) and (2). For the HTI rock, the minimum and maximum horizontal stresses using Eqs. (3) and (4).

For the rock containing a set of inclined fractures, the VTI or HTI model for horizontal stress calculation may not be applied directly. According to one embodiment of the disclosure, an integrated method which combines the VTI and the HTI models is used to determine the minimum horizontal stress. When the dip angle of the fractures varies from 0 to 90 degrees, it is assumed that the minimum horizontal stress changes gradually from the VTI model to the HTI model. In addition, a linear interpolation to take into account this change from the VTI model (Eqs. (1) and (2)) to the HTI model (Eqs. (3) and (4)). Specifically, if the dip angle of the fractures is 0, the VTI model is used; if the dip angle of the fractures is 90 degrees, the HTI model is used; if the dip angle of the fractures is in between of 0 to 90 degrees, a linear interpolation method is used to calculate the minimum stress, such as in Eq. (5). According to this approach and assuming σₕ=α_{V}=α, the minimum horizontal stress in the anisotropic case can be approximately obtained by combining Eq. (1) and Eq. (3):

In Step 706, comparing the calculated minimum and maximum horizontal stresses to the measured values.

In Step 707, if the difference between each calculated horizontal stress and the measured value is less than the threshold value, then the calculated result is acceptable. Otherwise, go to step 708.

In Step 708, the tectonic strain value is updated and used in Step 705 to recalculate the minimum and maximum horizontal stresses.

Variations of Eqs. (1)-(4) are available by simplifying the terms and coefficients to various degrees as the conditions permit. For example, the Biot's coefficients in the horizontal direction (an) and in the vertical directions (α_{V}) may be considered the same. Piosson's ratios in the horizontal direction and in the vertical direction may be treated the same way. Further, when the rock containing a set of inclined fractures, the VTI or HTI model for horizontal stress calculation may not be applied directly. an integrated method, combining the VTI and the HTI models, such as Eq. (5) may be employed when the fractures are dipping in different angles:

$$( 5 )$$

$${σ}_{{h}_{TI}} \approx \frac{β + ( 90 - β ) {B}^{2}}{90 B} \frac{v}{1 - v} ( {σ}_{V} - {p}_{p} ) + {p}_{p} + \frac{β + ( 90 - β ) A}{90 A} c ( {σ}_{V} - {p}_{p} ) .$$

In Eq. (5), β is the dip angle of the inclined fractures (degree), and for the horizontal fracture β=0, for the vertical fracture

$$β = 90 ° ; B = \frac{A}{{C}_{A}} , {C}_{A} = {C}_{V} + \frac{{C}_{H} - {C}_{V}}{90} β , {C}_{B} = \frac{90 + ( {C}_{H} - 1 ) β}{90} ,$$

C_{H}=v_{Vh}/v, and C_{V}=v/v_{V}. Further, A is a parameter that describes the anisotropy of Young's modulus.

$$A = 1 + \frac{E}{{k}_{n} s} ,$$

in which kₙ is a fracture normal stiffness and s is the fracture spacing. Parameter A increases as the fracture density increases and as the fracture stiffness decreases. Both kₙ and s are obtained using well log data.

However, when laboratory characterization of the rock specimen is available and accurate, Eqs. (1)-(4) may provide the most accurate in situ stress determination for VTI and HTI rocks.

### EXAMPLES

Validation of a method in this disclosure was carried out using measured data in the Xujiahe gas reservoir in China.

Image logging results in more than dozen wells in the Xujiahe gas reservoirs in the studied area show that the Xu 2 sandstone reservoir contains many natural fractures with different dip angles. The density of the natural fractures increases as the fracture dip angle increases, and most natural fractures are fractures with high dip angles.

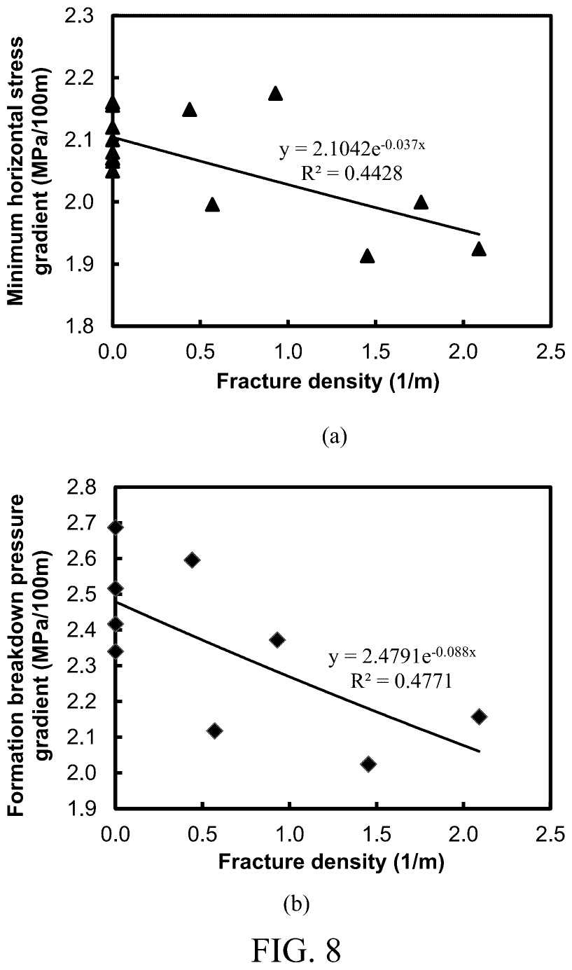

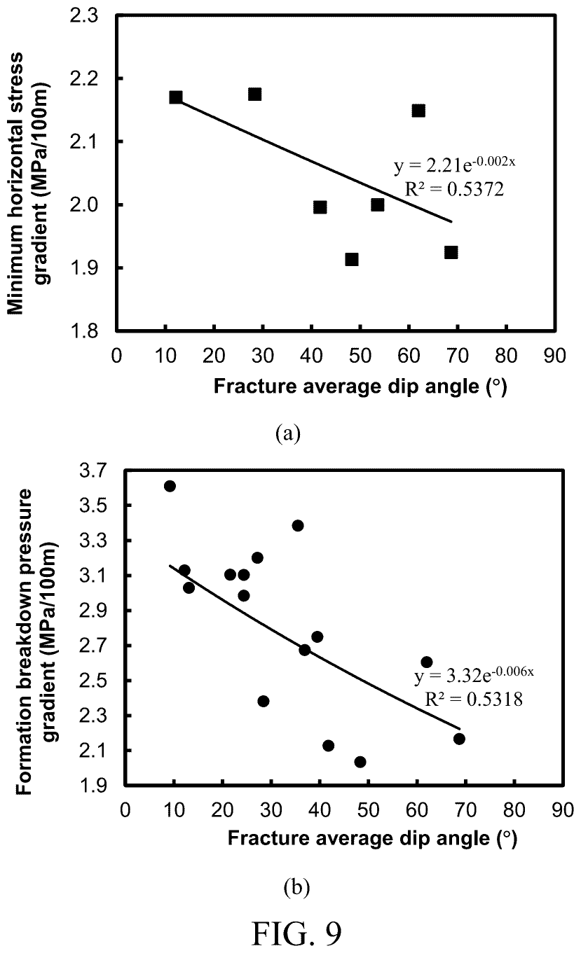

FIGS. 8 and 9 present the natural fracture impacts on the minimum horizontal stresses and breakdown pressures in 10 wells, which have both natural fracture data, measured results of the minimum horizontal stresses and formation breakdown pressures. FIG. 8, panel (a) shows that the trend of the measured minimum horizontal stress gradient decreases as the fracture density increases. FIG. 8, panel (b) indicates that the trend of the measured formation breakdown pressure gradient also decreases as the fracture density increases.

FIG. 9 shows that the fracture dip angles have significant impacts on the minimum horizontal stress gradient and formation breakdown pressure gradient. For example, the minimum horizontal stress gradient and formation breakdown pressure gradient decrease as the fracture dip angle increases, as shown in FIG. 9. FIG. 8, panel (a) and FIG. 9, panel (a) indicate that highly inclined natural fractures can result in a decrease of the minimum horizontal stress by up to 3 MPa/km. This is consistent with the HTI model since when the fracture dip angle increases, the naturally fractured rock approaches the HTI rock which has a smaller horizontal stress. The natural fractures FIGS. 8 and 9 were interpreted from image logs, and the minimum horizontal stress data were interpreted from DFIT tests.

FIG. 8, panel (b) and FIG. 9, panel (b) also indicate that formation breakdown pressure decreases as the fracture density and dip angle increase, and natural fractures can cause a decrease of the breakdown pressure by up to 10 MPa/km (or 50 MPa at depth of 5000 m), a much larger decrease than the minimum horizontal stress decrease. Therefore, the naturally fractured formation is much easier to be hydraulically fractured than the intact rock.

Dynamic Young's moduli, Poisson's ratios, and bulk moduli in the transversely isotropic rock can be obtained from the acoustic velocities. The dynamic data then need to be converted to the static data to apply the transversely isotropic models (VTI and HTI) according to Eqs. (1)-(4) or the anisotropic model, e.g., Eq. (5) to calculate the minimum horizontal stresses. However, conversion from dynamic to static rock properties using empirical equations is often inaccurate.

Static rock properties that are obtained from laboratory tests. If these rock properties are difficult to obtain, then empirical equations can be used to estimate horizontal stresses in the anisotropic rocks. Based on the measured data of natural fractures and the minimum horizontal stresses in the studied area, empirical equations of the minimum horizontal stress for this study:

$${σ}_{h , f} = {e}^{- 0.037 n} {σ}_{h} , \qquad ( 6 )$$

$${σ}_{h , f} = {e}^{- 0.002 β} {σ}_{h} . \qquad ( 7 )$$

In Eqs. (6) and (7) σ_{h,f} is the minimum horizontal stress in the naturally fractured rock (MPa); n is the density of the fractures (1/m); σₕ is the minimum horizontal stress in the isotropic rock (MPa), which can be obtained from existing method.

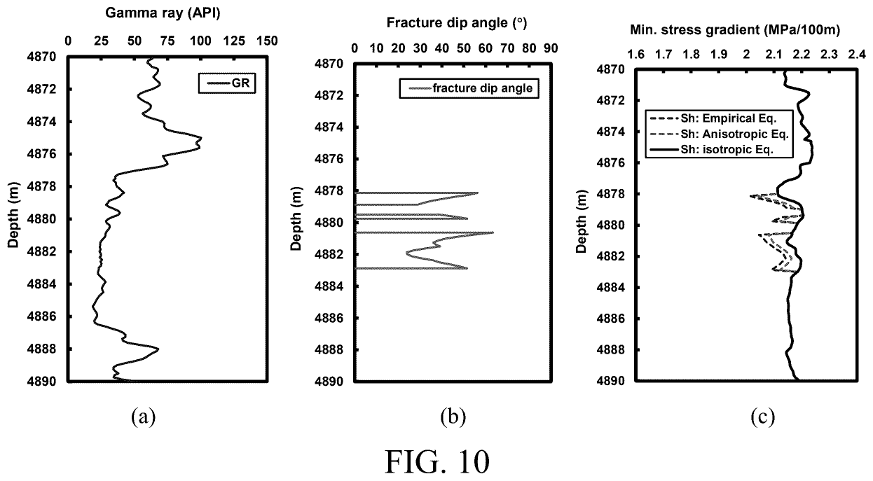

FIG. 10 shows an example of the application in Well Xin-10 in the studied area. In FIG. 10, panel (c) illustrates the comparisons of the minimum horizontal stress estimations from empirical equation, the anisotropic method (Eq. 5) and the isotropic model. Gamma ray log data in this well (FIG. 10, panel (a)) show that the Xu2 sandstone reservoir located at depth of 4877 to 4887m is a naturally fractured gas-bearing formation. The dip angles of the natural fractures in the reservoir are interpreted from well log data and plotted in panel (b), and the natural fractures have different dip angles varying from 24° to 63°.

The minimum horizontal stresses are estimated from the fracture dip angle correlation, the the anisotropic model, and the isotropic method, which are plotted in panel (c) for comparison. In the anisotropic model of Eq. (5), pore pressure (pₚ) is obtained from the measured results. Poisson's ratio (v) is calculated from Vₚ and Vₛ from sonic log data, and parameters A=1.438, C_{H}=C_{V}=1.37, c=0.45, α=1 are used for calculations. It can be seen from panel (c) that the proposed anisotropic model estimates a smaller minimum horizontal stress than the isotropic model because of presence of inclined natural fractures. The minimum horizontal stress reduction caused by high dip-angle fractures can reach 6%. This stress reduction is beneficial for hydraulic fracturing operations, because it causes formation breakdown pressure to decrease. Therefore, rock anisotropic effects need to be considered for in-situ stress estimate. FIG. 10, panel (c) also shows that the calculated result from proposed anisotropic model according to Eq. (5) is consistent to the one from the empirical equation. This implies that the proposed anisotropic model is applicable. The same conclusion is obtained from other wells in the studied area.

The example demonstrates that methods according to the instant disclosure are applicable in a range of conditions, whether rock properties can be reliably measured or not.

While embodiments of this disclosure have been shown and described, modifications can be made by one skilled in the art without departing from the spirit or teaching of this invention. The embodiments described herein are exemplary only and are not limiting. Many variations and modifications of methods, systems and apparatuses are possible and are within the scope of the invention. Accordingly, the scope of protection is not limited to the embodiments described herein, but is only limited by the claims. The scope of the claims shall include all equivalents of the subject matter of the claims.

We claim:

1. A method for determining horizontal stress in a subsurface anisotropic rock, comprising the steps of:
S0: investigating the anisotropic rock by one or more means selected from well logging, seimic survey, and core sampling to obtain one or more properties of the anisotropic rock;
S1: calculating a vertical stress based on a bulk density of the anisotropic rock;
S2: calculating a rock property based on well log data or seismic survey, wherein the rock property is one or more selected from pore pressure, fracture normal stiffness, the fracture spacing;
S3: conducting laboratory testing of a core sample of the anisotropic rock to obtain parameters including Young's modulus, Poisson's ratio, and thermal expansion coefficient, and tectonic strain;
S4: determining a dip angle of factures in the anisoptropic rock; and
S5: calculating the minimum horizontal stress and/or the maximum horizontal stress in the anisotropic rock according to the dip angle in the anisotropic rock.

2. The method according to claim 1, wherein, when the dip angle is 0°, calculating the minimum horizontal stress according to Eq. (1), and calculating the maximum horizontal stress according to Eq. (2):
$${σ}_{h \_ VTI} = \frac{{E}_{h} {v}_{V}}{{E}_{V} ( 1 - {v}_{h} )} ( {σ}_{V} - {α}_{V} {p}_{p} ) + {α}_{h} {p}_{p} + \frac{{E}_{h}}{1 - {v}_{h}^{2}} ( {ε}_{h} + {v}_{h} {ε}_{H} ) + \frac{{E}_{h} {α}_{Th}}{1 - {v}_{h}} Δ T , \qquad ( 1 )$$
$${σ}_{H \_ VTI} = \frac{{E}_{h} {v}_{V}}{{E}_{V} ( 1 - {v}_{h} )} ( {σ}_{V} - {α}_{V} {p}_{p} ) + {α}_{h} {p}_{p} + \frac{{E}_{h}}{1 - {v}_{h}^{2}} ( {ε}_{H} + {v}_{h} {ε}_{h} ) + \frac{{E}_{h} {α}_{Th}}{1 - {v}_{h}} Δ T , \qquad ( 2 )$$
wherein σ_{h_VTI} and σ_{H_VTI} are the minimum and maximum horizontal stresses, respectively; E_{V} and v_{V} are static Young's modulus and Poisson's ratio in the vertical direction, respectively; Eₕ and vₕ are static Young's modulus and Poisson's ratio in the horizontal direction, respectively; σ_{V} is the vertical stress; pₚ is the pore pressure; αₕ and α_{V} are Biot's coefficients in the horizontal and vertical directions, respectively; εₕ and ε_{H} are tectonic strains in the minimum and maximum horizontal stress directions, respectively; α_{Th} is the thermal expansion coefficient in the horizontal direction; and ΔT is a temperature difference in a burial history in the anisotropic rock.

3. The method according to claim 2, wherein, when the dip angle is 90°, calculating the minimum horizontal stress according to Eq. (3), and calculating the maximum horizontal stress according to Eq. (4):
$${σ}_{h \_ HTI} = \frac{{E}_{h} {v}_{Vh} ( 1 + {v}_{VH} )}{{E}_{V} ( 1 - {v}_{Vh} {v}_{hV} )} ( {σ}_{V} - {α}_{V} {p}_{p} ) + {α}_{h} {p}_{p} + \frac{{E}_{h} ( {ε}_{h} + {v}_{Vh} {ε}_{H} )}{1 - {v}_{Vh} {v}_{hV}} + \frac{{E}_{h} ( {α}_{Th} + {v}_{Vh} {α}_{TH} )}{1 - {v}_{Vh} {v}_{hV}} Δ T , \qquad ( 3 )$$
$${σ}_{H \_ HTI} = \frac{{v}_{VH} + {v}_{Vh} {v}_{hV}}{1 - {v}_{Vh} {v}_{hV}} ( {σ}_{V} - {α}_{V} {p}_{p} ) + {α}_{V} {p}_{p} + \frac{{E}_{V} ( {ε}_{H} + {v}_{hV} {ε}_{h} )}{1 - {v}_{Vh} {v}_{hV}} + \frac{{E}_{V} ( {α}_{TH} + {v}_{hV} {α}_{Th} )}{1 - {v}_{Vh} {v}_{hV}} Δ T , \qquad ( 4 )$$
wherein σ_{h_HTI} and σ_{H_HTI} are the minimum and maximum horizontal stresses in the HTI rock, respectively; E_{V} and Eₕ are static Young's moduli in the vertical and horizontal directions, respectively; v_{Vh}, v_{VH}, and v_{hV} are static Poisson's ratios; v_{hH}=v_{hV}, v_{HV}=v_{VH}, and v_{Vh}=v_{Hh}, and vₕᵥ/Eₕ=v_{Vh}/E_{V}; and α_{Th} and α_{TH} are thermal expansion coefficients in the minimum and maximum horizontal stress directions, respectively.

4. The method of claim 3, wherein, when the dip angle is between 0° and 90°, calculating the minimum horizontal stress of the anisotropic rock according to Eq. (5):
$$( 5 )$$
$${σ}_{{h}_{TI}} \approx \frac{β + ( 90 - β ) {B}^{2}}{90 B} \frac{v}{1 - v} ( {σ}_{V} - {p}_{p} ) + {p}_{p} + \frac{β + ( 90 - β ) A}{90 A} c ( {σ}_{V} - {p}_{p} ) ,$$
wherein β is the dip angle,
$$B = \frac{A}{{C}_{A}} , {C}_{A} = {C}_{V} + \frac{{C}_{H} - {C}_{V}}{90} β , {C}_{B} = \frac{90 + ( {C}_{H} - 1 ) β}{90} ,$$
C_{H}=v_{Vh}/v, C_{V}=v/v_{V}, and
$$A = 1 + \frac{E}{{k}_{n} s} ,$$
wherein kₙ is a fracture normal stiffness and s is a fracture spacing.

5. The method of claim 1, further comprising:
S6: obtaining a measured maximum horizontal stress and a measured maximum horizontal stress; and comparing the measured minimum horizontal stress with the calculated minimum horizontal stress to obtain a first difference and/or comparing the measured maximum horizontal stress with the calculated maximum horizontal stress to obtain a second difference; and
S7: when the first difference or the second difference exceeds a threshold value, adjusting a value of the tectonic strain; otherwise, outputting the calculated minimum horizontal stress and the calculated maximum horizontal stress as true; and
S8: repeating S5 to S7 until the first difference and/or the second difference is at or below the threshold value.

6. The method of claim 1, further comprising:
converting dynamic the rock property obtained from well logging or seismic survey to a static rock property.
